(19) United States
(12) Patent Application Publication
(10) Pub. No.: US 2026/0056175 A1
(43) Pub. Date: Feb. 26, 2026

(54) ONLINE MONITORING SYSTEM FOR FLUE GAS CARBON DIOXIDE EMISSIONS

(71) Applicant: DATANG ENVIRONMENT INDUSTRY GROUP COL., LTD., Beijing (CN)

(72) Inventors: Mingsheng DU, Beijing (CN); Yuyong BAI, Beijing (CN); Lei MENG, Beijing (CN); Xiao CHONG, Beijing (CN); Yuyu LI, Beijing (CN); Siwei PENG, Beijing (CN); Peng ZHANG, Beijing (CN)

(73) Assignee: DATANG ENVIRONMENT INDUSTRY GROUP COL., LTD., Beijing (CN)

(21) Appl. No.: 18/873,194

(22) PCT Filed: Jul. 26, 2024
(86) PCT No.: PCT/CN2024/107757

§ 371 Date: Mar. 28, 2025

(30) Foreign Application Priority Data
Jun. 13, 2024 (CN) 202410762313.4

(51) Int. Cl. G01N 33/00; G01F 1/46

(52) U.S. Cl. CPC G01N 33/0027; G01F 1/46

(57) ABSTRACT

an online monitoring for flue gas carbon dioxide emissions is provided by the present disclosure, including a standard grid method flow analyzer, a CO2 concentration analyzer, a temperature-pressure-humidity analyzer and a CO2 emission online monitoring platform. The standard grid method flow analyzer includes a standard grid method flow analyzer cabinet and two sets of flue gas multipoint flow rate sampling probes fixedly connected with flue walls on both sides of a flue so that measurement of flue gas CO2 flow rate within the flue is distributed in a grid format. A sampling probe of the CO2 concentration analyzer projects into the flue, a sampling probe of the temperature-pressure-humidity analyzer projects into the flue, and the standard grid method flow analyzer cabinet, the CO2 concentration analyzer and the temperature-pressure-humidity analyzer are electrically connected with the online monitoring platform 7 for CO2 emission, respectively. The present disclosure can realize low hysteresis, high accuracy and high reliability of online monitoring for flue gas CO2 emission.

### FIELD OF THE INVENTION

The disclosure relates to the technical field of carbon dioxide monitoring and in particular to an online monitoring for flue gas carbon dioxide emissions.

### BACKGROUND OF THE INVENTION

At present, the emission monitoring of carbon dioxide in flue gas is one of the important means of environmental protection. Accurate and real-time monitoring of carbon dioxide in flue gas is of great significance to robust carbon emission metering management and carbon emission regulation.

In order to to reduce carbon dioxide emissions in the atmosphere, a series of policies and measures have been introduced at home and abroad in recent years to control carbon dioxide emissions. Currently there are two main methods for emission control of carbon dioxide, an accounting method and a measurement method. The accounting method calculates the total amount of emissions of carbon dioxide from raw material usage data, fuel combustion activity data and corresponding emission factors for each type of activity. The measurement method is to install an online monitoring device at a discharge end to obtain the total amount of carbon emissions by collecting data such as flue gas concentration, volumetric flow rate and the like. Because the accounting method involves a variety of activity data and emission factors, and there are many human disturbances, it has some problems such as low accuracy, large error, time lag and so on when monitoring carbon dioxide emissions from flue gas. With the development of measurement technology, the measurement method is able to avoid these problems of the accounting method.

Currently, the measurement of carbon dioxide concentration mainly adopts extraction measurement, where the carbon dioxide flue gas is first pre-treated and then the treated flue gas is drawn into a carbon dioxide analyzer for measurement. This sampling approach results in hysteresis of the carbon dioxide concentration measurement and does not reflect real carbon dioxide measurements in real time. Furthermore, because of the relatively wide flue on site and the uneven distribution of flue gas within the flue, the existing single point flow meter is also unable to accurately provide flow rate and flow data for carbon dioxide in flue gas on site when making flow measurements.

Since carbon dioxide concentration, flue gas flow rate and flue gas flow are the most critical parameters for measuring carbon dioxide emissions from flue gas, there is a great need to design an online monitoring system for carbon dioxide emission with low hysteresis while being capable of accurately measuring carbon dioxide concentration, flue gas flow rate and flue gas flow.

### SUMMARY OF THE INVENTION

It is an object of the present disclosure to provide an online monitoring for flue gas carbon dioxide emissions that enables low hysteresis, high accuracy and high reliability of flue gas carbon dioxide emission online monitoring.

The present disclosure provides an online monitoring for flue gas carbon dioxide emissions including:
a standard grid method flow analyzer including a standard grid method flow analyzer cabinet and two sets of flue gas multipoint flow rate sampling probes electrically connected with the standard grid method flow analyzer cabinet, in which the two sets of flue gas multipoint flow rate sampling probes are respectively disposed within a flue and are fixedly connected with flue walls on both sides of the flue, so that measurement of flue gas CO2 flow rate within the flue is distributed in a grid format;
a CO2 concentration analyzer mounted on the outside of the flue wall with a sampling probe of the CO2 concentration analyzer projecting into the flue;
a temperature-pressure-humidity analyzer mounted on the outside of the flue wall with a sampling probe of the temperature-pressure-humidity analyzer projecting into the flue; and
a CO2 emission online monitoring platform disposed outside the flue, in which the standard grid method flow analyzer cabinet, the CO2 concentration analyzer and the temperature-pressure-humidity analyzer are electrically connected with the online monitoring platform 7 for CO2 emission, respectively.

According to the present disclosure, there is provided an online monitoring for flue gas carbon dioxide emissions further including a power distribution box, in which the standard grid method flow analyzer cabinet, the CO2 concentration analyzer and the temperature-pressure-humidity analyzer are electrically connected with the power distribution box, respectively.

According to the present disclosure, there is provided an online monitoring for flue gas carbon dioxide emissions, in which the CO2 concentration analyzer includes a CO2 concentration analyzer cabinet, a CO2 concentration sampling probe, an optical measurement cell, an optical measurement module, a jet pump, a first power supply module, a heating module, a first control module, a photoelectric conversion module and a signal output module.

The CO2 concentration analyzer cabinet is secured to the outside of the flue wall by a first mounting flange, and the optical measurement cell, the optical measurement module, the jet pump, the first power supply module, the heating module, the first control module, the photoelectric conversion module and the signal output module are respectively disposed in the CO2 concentration analyzer cabinet. The first power supply module is connected with the first control module, the first control module is connected with the heating module, the jet pump is connected with the optical measuring cell, the optical measurement module is connected with the optical measurement cell, the first control module is connected with the optical measurement module, the photoelectric conversion module is connected with the optical measurement module, the photoelectric conversion module is connected with the signal output module, and the signal output module is connected with the online monitoring platform for CO2 emission.

According to the present disclosure, there is provided an online monitoring for flue gas carbon dioxide emissions, in which the CO2 concentration sampling probe includes a sampling tube, a heating sleeve and a filter element. One end of the sampling tube is connected with the optical measuring cell, and the other end of the sampling tube extends through the flue wall into the flue. The filter element is disposed inside the sampling tube, and the heating sleeve is set on the outside of the sampling tube.

According to the present disclosure, there is provided an online monitoring for flue gas carbon dioxide emissions, in which each set of flue gas multipoint flow rate sampling probes is fixedly connected with the flue walls via a second mounting flange.

According to the present disclosure, there is provided an online monitoring for flue gas carbon dioxide emissions, in which a first solenoid valve set, a second solenoid valve set, a first differential pressure transmitter, a second differential pressure transmitter, a second power supply module, a second control module and a data output module respectively are provided within the standard grid method flow analyzer cabinet.

The first solenoid valve set is connected with one set of flue gas multipoint flow rate sampling probes via a gas line, and the first differential pressure transmitter is connected with the first solenoid valve set via a gas line. The second solenoid valve set is connected with another set of the flue gas multipoint flow rate sampling probes via a gas line, and the second differential pressure transmitter is connected with the second solenoid valve set via a gas line. The second power supply module is connected with the second control module, the first solenoid valve set and the second solenoid valve set are respectively connected with the second control module. The first differential pressure transmitter and the second differential pressure transmitter are respectively connected with the second control module, the first differential pressure transmitter and the second differential pressure transmitter are respectively connected with the data output module, and the data output module is connected with the online monitoring platform for CO2 emission.

According to the present disclosure, there is provided an online monitoring for flue gas carbon dioxide emissions, in which the two sets of flue gas multipoint flow rate sampling probes are symmetrically distributed, wherein each set of flue gas multipoint flow rate sampling probes includes 5 pitot tubes, which are spaced apart in sequence from a position proximate the flue wall to a position proximate a center of the flue, and spacing between each adjacent two pitot tubes increases in sequence from outside to inside.

According to the present disclosure, there is provided an online monitoring for flue gas carbon dioxide emissions, in which each of the pitot tubes includes a full pressure sampling tube and a static pressure sampling tube. The full pressure sampling tube includes a full pressure sampling tube body and a full pressure sampling port disposed at a tip of the full pressure sampling tube body. The static pressure sampling tube includes a static pressure sampling tube body and a static pressure sampling port disposed at a tip of the static pressure sampling tube body. An outer sidewall of the full pressure sampling tube body and an outer sidewall of the static pressure sampling tube body are bonded to each other, and the full pressure sampling port is disposed opposite to the static pressure sampling port.

According to the present disclosure, there is provided an online monitoring for flue gas carbon dioxide emissions, in which the first solenoid valve set includes a first full pressure sampling tube solenoid valve set and a first static pressure sampling tube solenoid valve set, and the second solenoid valve set includes a second full pressure sampling tube solenoid valve set and a second static pressure sampling tube solenoid valve set. The first full pressure sampling tube solenoid valve set is connected with the full pressure sampling tube of one set of flue gas multipoint flow rate sampling probes, and the second full pressure sampling tube solenoid valve set is connected with the full pressure sampling tube of another set of flue gas multipoint flow rate sampling probes. The first static pressure sampling tube solenoid valve set is connected with the static pressure sampling tube of one set of flue gas multipoint flow rate sampling probes, and the second static pressure sampling tube solenoid valve set is connected with the static pressure sampling tube of another set of flue gas multipoint flow rate sampling probes.

According to the present disclosure, there is provided an online monitoring for flue gas carbon dioxide emissions, in which the CO2 concentration analyzer is an in-situ hot moisture CO2 concentration analyzer, and the temperature-pressure-humidity analyzer is an integrated temperature-pressure-humidity analyzer.

According to the present disclosure, there is provided an online monitoring for flue gas carbon dioxide emissions, in which the standard grid method flow analyzer, the CO2 concentration analyzer and the temperature-pressure-humidity analyzer are capable of sampling the flue gas CO2 within the flue, respectively. The standard grid method flow analyzer, the CO2 concentration analyzer and the temperature-pressure-humidity analyzer are then capable of measuring the sampled flue gas CO2 within the flue, respectively. The measured data is finally transmitted to the online monitoring platform CO2 emission, thereby enabling online monitoring for flue gas carbon dioxide emissions. Thus, the present disclosure provides an online monitoring for flue gas carbon dioxide emissions that enables low hysteresis, high accuracy and high reliability of the online monitoring for the flue gas carbon dioxide emission.

### BRIEF DESCRIPTION OF THE DRAWINGS

In order to more clearly illustrate embodiments of the present disclosure or the technical solutions in the prior art, the accompanying drawings used in the description of the embodiments will now be briefly described. It will be apparent that the drawings in the following description are some embodiments of the present disclosure and that other drawings can be derived from these drawings without inventive step for a person of ordinary skill in the art.

FIG. 1 is a schematic diagram of an online monitoring system for flue gas carbon dioxide emission according to the present disclosure;

FIG. 2 is a schematic diagram of a CO2 concentration analyzer in an online monitoring system for flue gas carbon dioxide emission according to the present disclosure;

FIG. 3 is a schematic diagram of a standard grid method flow analyzer in an online monitoring system for flue gas carbon dioxide emission according to the present disclosure;

FIG. 4 is a schematic diagram of a pitot tube in an online monitoring system for flue gas carbon dioxide emission according to the present disclosure; and

FIG. 5 shows a monitoring point layout of two sets of flue gas multipoint flow rate sampling probes in the online monitoring system for flue gas carbon dioxide emission according to the present disclosure.

### DETAILED DESCRIPTION OF THE INVENTION

Technical solutions of the present disclosure will now be clearly and completely described with reference to embodiments and it will be apparent that the described embodiments are some, but not all, of embodiments of the disclosure. Based on the embodiments of the present disclosure, all other embodiments obtained by a person of ordinary skill in the art without inventive step are within the scope of protection of the present disclosure.

In the description of the disclosure, it needs to be understood that the terms “central,” “longitudinal,” “lateral,” “length,” “width,” “thickness,” “upper,” “lower,” “front,” “back,” “left,” “right,” “vertical,” “horizontal,” “top,” “bottom,” “inner,” “outer,” “clockwise,” “counterclockwise,” and the like indicate an orientation or positional relationship based on that shown in the drawings. It is merely for convenience of description of the disclosure and simplification of the description. It is not intended to indicate or imply that the devices or elements referred to must have a particular orientation, be constructed and operate in a particular orientation and therefore cannot be construed as limiting the present disclosure.

Furthermore, the terms “first” and “second” are used for descriptive purposes only and are not to be understood as indicating or implying relative importance or as implying a number of indicated technical features. Thus, features qualified as “first” and “second” may explicitly or implicitly include one or more of the features. In the description of the present disclosure, “plurality” means two or more than two, unless specifically defined otherwise. Furthermore, the terms “mounted” and “connected” are to be construed broadly and may be, for example, fixedly or removably connected, or integrally connected, either mechanically or electrically. The connection may be direct or indirect via an intermediary and the communication may be internal to the two elements. The specific meaning of the above terms in the present disclosure can be understood, on a case-by-case basis, by those of ordinary skill in the art.

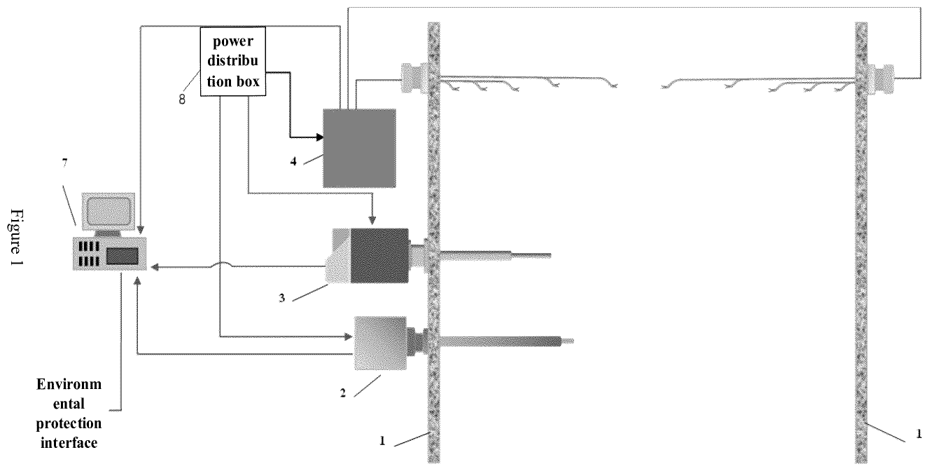

As shown in FIG. 1, an online monitoring for flue gas carbon dioxide emissions according to an embodiment of the present disclosure includes a standard grid method flow analyzer, a CO2 concentration analyzer 3, a temperature-pressure-humidity analyzer 2 and an online monitoring platform 7 for CO2 emission.

Herein, the standard grid method flow analyzer includes a standard grid method flow analyzer cabinet 4 and two sets of flue gas multipoint flow rate sampling probes electrically connected with the standard grid method flow analyzer cabinet 4. The two sets of flue gas multipoint flow rate sampling probes are a first set of flue gas multipoint flow rate sampling probes and a second set of flue gas multipoint flow rate sampling probes, respectively. The two sets of flue gas multipoint flow rate sampling probes are disposed within a flue and are fixedly connected with both sides of a flue wall 1, such that the measurement of flue gas CO2 flow rate within the flue is distributed in a grid format.

The CO2 concentration analyzer 3 is mounted outside the flue wall 1, and a sampling probe of the CO2 concentration analyzer 3 is projected into the flue.

The temperature-pressure-humidity analyzer 2 is mounted outside the flue wall 1, and a sampling probe of the temperature-pressure-humidity analyzer 2 is projected into the flue.

The online monitoring platform 7 for CO2 emission is disposed outside the flue, and the standard grid method flow analyzer cabinet 4, the CO2 concentration analyzer 3 and the temperature-pressure-humidity analyzer 2 are electrically connected with the online monitoring platform 7 for CO2 emission, respectively.

That is, the standard grid method flow analyzer, the CO2 concentration analyzer 3 and the temperature-pressure-humidity analyzer 2 are capable of sampling the flue gas CO2 within the flue, respectively. Then, the standard grid method flow analyzer, the CO2 concentration analyzer 3 and the temperature-pressure-humidity analyzer 2 are capable of measuring the sampled flue gas CO2 within the flue, respectively. The measured data is finally transmitted to the online monitoring platform 7 for CO2 emission, thereby enabling online monitoring for flue gas carbon dioxide emission. Thus, the embodiment of the present disclosure provides an online monitoring for flue gas carbon dioxide emissions that enables low hysteresis, high accuracy and high reliability of the online monitoring for flue gas carbon dioxide emissions.

Further, the online monitoring for flue gas carbon dioxide emissions system may include a power distribution box 8, and the standard grid method flow analyzer cabinet, the CO2 concentration analyzer 3 and the temperature-pressure-humidity analyzer 2 are electrically connected with the power distribution box, respectively.

Specifically, the CO2 concentration analyzer 3 is an in-situ hot moisture CO2 concentration analyzer, and the temperature-pressure-humidity analyzer 2 is an integrated temperature-pressure-humidity analyzer.

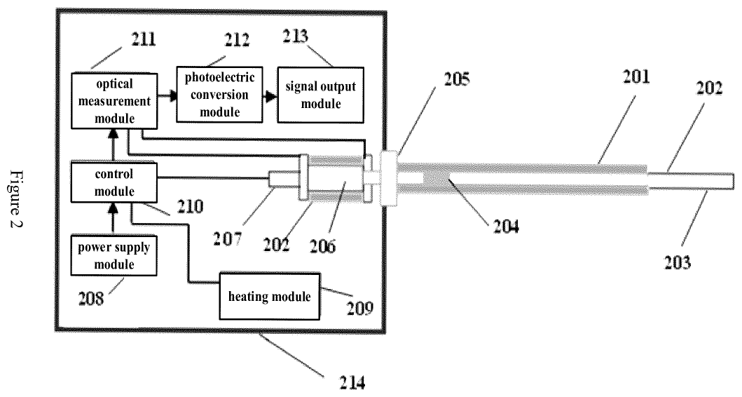

As shown in FIG. 2, the in-situ hot moisture CO2 concentration analyzer 3 includes an in-situ hot moisture sampling probe 201, a first mounting flange 205, an optical measurement cell 206, a jet pump 207, a first power supply module 208, a heating module 209, a first control module 210, an optical measurement module 211, a photoelectric conversion module 212, a signal output module 213 and a CO2 concentration analyzer cabinet 214. Herein, the in-situ hot moisture sampling probe 201 includes a heating sleeve 202, an in-situ hot moisture sampling tube 203 and a filter element 204.

The first mounting flange 205 is used to secure the CO2 concentration analyzer cabinet 214 to the flue wall 1. The optical measuring cell 206, the optical measuring module 211, the jet pump 207, the first power supply module 208, the heating module 209, the first control module 210, the photoelectric conversion module 212 and the signal output module 213 are disposed in the CO2 concentration analyzer cabinet 214, respectively. The first power supply module 208 is connected with the first control module 210 for powering the first control module 210. The first control module 210 is connected with the heating module 209. After the CO2 concentration analyzer 3 is started, the first control module 210 controls the heating module 209 to heat the heating sleeve 202 of the in-situ hot moisture sampling probe 201 and the heating sleeve 202 of the optical measuring cell 206, respectively. The jet pump 207 is connected with the optical measuring cell 206. After the heating temperatures of the in-situ hot moisture sampling probe 201 and the optical measuring cell 206 have been stabilized, the jet pump 207 draws the flue gas of the in-situ hot moisture sampling probe 201 into the optical measuring cell 206 under the control of the first control module 210. The optical measuring module 211 is connected with the optical measuring cell 206, and the first control module 210 is connected with the optical measuring module 211. The first control module 210 controls the optical measuring module 211 to measure the concentration of the flue gas CO2 drawn into the optical measuring cell 206. The photoelectric conversion module 212 is connected with the optical measurement module 211 to convert an optical signal of the CO2 concentration measured by the optical measurement module 211 into an electrical signal. The signal output module 213 is connected with the photoelectric conversion module 212, and is used to output the converted signal to the online monitoring platform 7 for CO2 emission in FIG. 1.

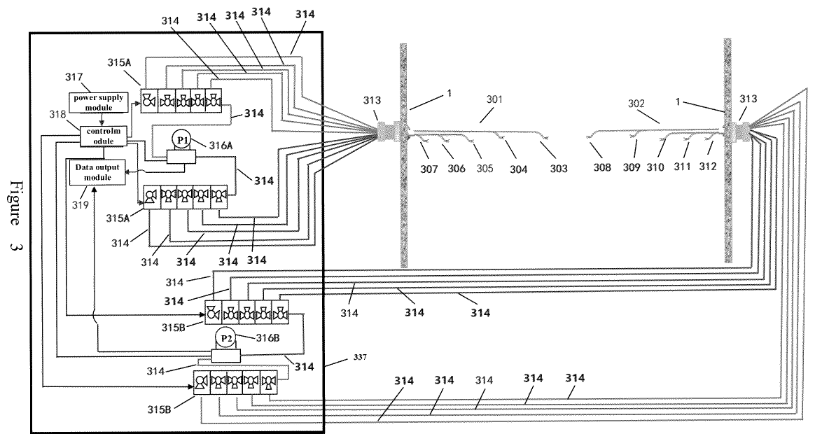

As shown in FIG. 3, the standard grid method flow analyzer includes two sets of flue gas multipoint flow rate sampling probes, two second mounting flanges 313, 24 gas lines 314, a first solenoid valve set 315A, a second solenoid valve set 315B, a first differential pressure transmitter 316A, a second differential pressure transmitter 316B, a second power supply module 317, a second control module 318 and a data output module 319. The two sets of flue gas multipoint flow rate sampling probes are a first set of flue gas multipoint flow rate sampling probes 301 and a second set of flue gas multipoint flow rate sampling probes 302, respectively. The two second mounting flanges 313 mount the two sets of flue gas multipoint flow rate sampling probes on both flue walls 1, respectively.

Specifically, the first set of flue gas multipoint flow rate sampling probes 301 and the second set of flue gas multipoint flow rate sampling probes 302 are symmetrically distributed, in which each set of flue gas multipoint flow rate sampling probes includes 5 pitot tubes, forming 10 probe points in total. Herein, the first set of flue gas multipoint flow rate sampling probes 301 includes a first pitot tube 303, a second pitot tube 304, a third pitot tube 305, a fourth pitot tube 306 and a fifth pitot tube 307 arranged in sequence from the center of the flue to the left flue wall. The second set of flue gas multipoint flow rate sampling probes 302 includes a sixth pitot tube 308, a seventh pitot tube 309, an eighth pitot tube 310, a ninth pitot tube 311 and a tenth pitot tube 312 arranged in sequence from the center of the flue to the right flue wall. As shown in FIG. 5, five pitot tubes of each set of flue gas multipoint flow rate sampling probes are sequentially spaced apart from a location near the flue wall to a location near the center of the flue and the spacing between each adjacent two pitot tubes increases sequentially from outside to inside. 10 dots in FIG. 5 are the 10 measure points of the two sets of flue gas multipoint flow rate sampling probes inside the flue. Hereby, a grid format multipoint measurement of flue gas CO2 flow rate within the flue can be achieved by 10 measure points through two sets of flue gas multipoint flow rate sampling probes.

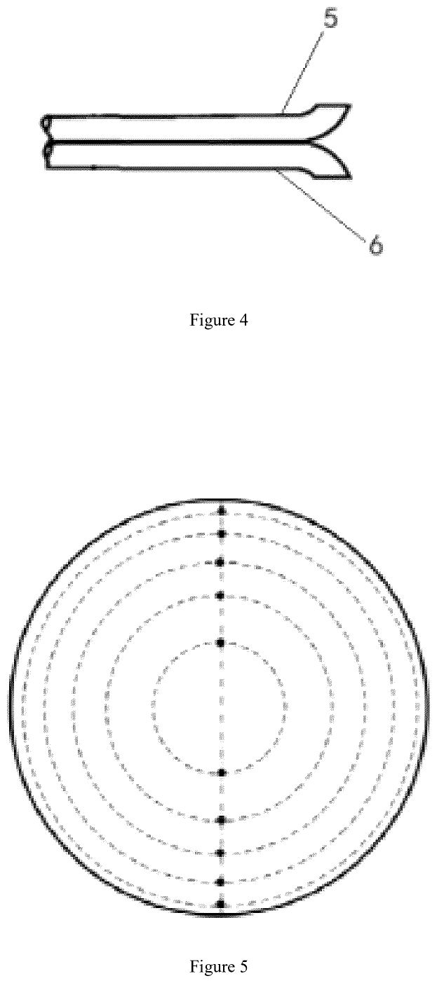

As shown in FIG. 4, each pitot tube includes a static pressure sampling tube 5 and a full pressure sampling tube 6. The static pressure sampling tube 5 includes a static pressure sampling tube body and a static pressure sampling port disposed at a tip of the static pressure sampling tube body. The full pressure sampling tube 6 includes a full pressure sampling tube body and a full pressure sampling port disposed at a tip of the full pressure sampling tube body. An outer sidewall of the full pressure sampling tube body and an outer sidewall of the static pressure sampling tube body are bonded to each other, and the full pressure sampling port is disposed opposite to the static pressure sampling port. Herein, the static pressure sampling port of the static pressure sampling tube 5 is disposed away from the flue gas flow direction, and the full pressure sampling port of the full pressure sampling tube 6 is disposed facing the flue gas flow direction.

As shown in FIG. 3, a second power supply module 317 is connected with the second control module 318 for supplying power to the second control module 318. The first solenoid valve set 315A includes a first full pressure sampling tube solenoid valve set and a first static pressure sampling tube solenoid valve set. The second solenoid valve set 315B includes a second full pressure sampling tube solenoid valve set and a second static pressure sampling tube solenoid valve set. Each full pressure sampling tube of the first flue gas multipoint flow rate sampling probe set 301 is connected with the first full pressure sampling tube solenoid valve set via a gas line, respectively. Each full pressure sampling tube of the second flue gas multipoint flow rate sampling probe set 302 is connected with the second full pressure sampling tube solenoid valve set via a gas line, respectively. Each static pressure sampling tube of the first flue gas multipoint flow rate sampling probe set 301 is connected with the first static pressure sampling tube solenoid valve set via a gas line, respectively. Each static pressure sampling tube of the second flue gas multipoint flow rate sampling probe set 302 is connected with the second static pressure sampling tube solenoid valve set via a gas line, respectively.

Herein, the first solenoid valve set 315A and the second solenoid valve set 315B are respectively connected with the second control module 318. The second control module 318 is used for controlling polling on/off of respective solenoid valves in the first solenoid valve set 315A and the second solenoid valve set 315B.

Herein, the first differential pressure transmitter 316A is connected with the first solenoid valve set 315A via a gas line. The second differential pressure transmitter 316B is connected with the second solenoid valve set 315B via a gas line. The first differential pressure transmitter 316A and the second differential pressure transmitter 316B are connected with the second control module 318, respectively. The flue gas CO2 taken through two sets of flue gas multipoint flow rate sampling probes enters the corresponding differential pressure transmitter through corresponding sets of solenoid valves, and the flue gas CO2 flow rate are measured under the control of the second control module 318.

The first differential pressure transmitter 316A and the second differential pressure transmitter 316B are respectively connected with the data output module 319, and the data output module 319 is connected with the online monitoring platform 7 for CO2 emission. The data output module 319 is configured to transmit the flue gas CO2 flow rate data measured by the two differential pressure transmitters to the online monitoring platform 7 for CO2 emission.

In summary, the online monitoring system for flue gas carbon dioxide emission according to embodiments of the present disclosure can combine in-situ hot moisture sampling technique with standard grid method plus polling differential pressure transmission flow rate measurement technique with the advantages of low hysteresis, high accuracy and high reliability, as compared to conventional online monitoring systems for CO2 emission.

Finally, it should be noted that the above embodiments are merely illustrative of the technical solutions of the present disclosure, instead of limiting them. Although the present disclosure has been described in detail with reference to the foregoing embodiments, it will be understood by those skilled in the art that the technical solutions described in the foregoing embodiments may be modified or equivalents may be substituted for some or all of the technical features thereof. Such modifications or substitutions do not cause the essence of the corresponding technical solutions to depart from the scope of the embodiments of the present disclosure.

1. An online monitoring for flue gas carbon dioxide emissions comprising:
a standard grid method flow analyzer comprising a standard grid method flow analyzer cabinet and two sets of flue gas multipoint flow rate sampling probes electrically connected with the standard grid method flow analyzer cabinet, wherein the two sets of flue gas multipoint flow rate sampling probes are respectively disposed within a flue and are fixedly connected with flue walls on both sides of the flue, so that measurement of flue gas CO2 flow rate within the flue is distributed in a grid format;
a CO2 concentration analyzer mounted on the outside of the flue wall with a sampling probe of the CO2 concentration analyzer projecting into the flue;
a temperature-pressure-humidity analyzer mounted on the outside of the flue wall with a sampling probe of the temperature-pressure-humidity analyzer projecting into the flue; and
a CO2 emission online monitoring platform disposed outside of the flue, wherein the standard grid method flow analyzer cabinet, the CO2 concentration analyzer and the temperature-pressure-humidity analyzer are electrically connected with the online monitoring platform 7 for CO2 emission, respectively.

2. The online monitoring for flue gas carbon dioxide emissions system according to claim 1, further comprising a power distribution box, wherein the standard grid method flow analyzer cabinet, the CO2 concentration analyzer and the temperature-pressure-humidity analyzer are electrically connected with the power distribution box, respectively.

3. The online monitoring for flue gas carbon dioxide emissions system according to claim 1, wherein the CO2 concentration analyzer comprises a CO2 concentration analyzer cabinet, a CO2 concentration sampling probe, an optical measurement cell, an optical measurement module, a jet pump, a first power supply module, a heating module, a first control module, a photoelectric conversion module and a signal output module, and
wherein the CO2 concentration analyzer cabinet is secured to the outside of the flue wall by a first mounting flange, and the optical measurement cell, the optical measurement module, the jet pump, the first power supply module, the heating module, the first control module, the photoelectric conversion module and the signal output module are respectively disposed in the CO2 concentration analyzer cabinet; wherein the first power supply module is connected with the first control module, the first control module is connected with the heating module, the jet pump is connected with the optical measuring cell, the optical measurement module is connected with the optical measurement cell, the first control module is connected with the optical measurement module, the photoelectric conversion module is connected with the optical measurement module, the photoelectric conversion module is connected with the signal output module, and the signal output module is connected with the online monitoring platform 7 for CO2 emission.

4. The online monitoring for flue gas carbon dioxide emissions system according to claim 3, wherein the CO2 concentration sampling probe comprises a sampling tube, a heating sleeve and a filter element, wherein one end of the sampling tube is connected with the optical measuring cell, the other end of the sampling tube extends through the flue wall into the flue, the filter element is disposed inside the sampling tube, and the heating sleeve is set on the outside of the sampling tube.

5. The online monitoring for flue gas carbon dioxide emissions system according to claim 1, wherein each set of flue gas multipoint flow rate sampling probes is fixedly connected with the flue walls via a second mounting flange, respectively.

6. The online monitoring for flue gas carbon dioxide emissions system according to claim 1, wherein a first solenoid valve set, a second solenoid valve set, a first differential pressure transmitter, a second differential pressure transmitter, a second power supply module, a second control module and a data output module are provided within the standard grid method flow analyzer cabinet, respectively, and
wherein the first solenoid valve set is connected with one set of flue gas multipoint flow rate sampling probes via a gas line, and the first differential pressure transmitter is connected with the first solenoid valve set via a gas line; wherein the second solenoid valve set is connected with another set of the flue gas multipoint flow rate sampling probes via a gas line, and the second differential pressure transmitter is connected with the second solenoid valve set via a gas line; wherein the second power supply module is connected with the second control module, the first solenoid valve set and the second solenoid valve sets are respectively connected with the second control module, the first differential pressure transmitter and the second differential pressure transmitter are respectively connected with the second control module, the first differential pressure transmitter and the second differential pressure transmitter are respectively connected with the data output module, and the data output module is connected with the online monitoring platform 7 for CO2 emission.

7. The online monitoring for flue gas carbon dioxide emissions system according to claim 6, wherein the two sets of flue gas multipoint flow rate sampling probes are symmetrically distributed; wherein each set of flue gas multipoint flow rate sampling probes comprises 5 pitot tubes, which are spaced apart in sequence from a position proximate the flue wall to a position proximate a center of the flue, and spacing between each adjacent two pitot tubes increases in sequence from outside to inside.

8. The online monitoring for flue gas carbon dioxide emissions system according to claim 7, wherein each of the pitot tubes comprises a full pressure sampling tube and a static pressure sampling tube, the full pressure sampling tube comprises a full pressure sampling tube body and a full pressure sampling port disposed at a tip of the full pressure sampling tube body, the static pressure sampling tube comprises a static pressure sampling tube body and a static pressure sampling port disposed at a tip of the static pressure sampling tube body, an outer sidewall of the full pressure sampling tube body and an outer sidewall of the static pressure sampling tube body are bonded to each other, and the full pressure sampling port is disposed opposite to the static pressure sampling port.

9. The online monitoring for flue gas carbon dioxide emissions system according to claim 8, wherein the first solenoid valve set comprises a first full pressure sampling tube solenoid valve set and a first static pressure sampling tube solenoid valve set, and the second solenoid valve set comprises a second full pressure sampling tube solenoid valve set and a second static pressure sampling tube solenoid valve set; wherein the first full pressure sampling tube solenoid valve set is connected with the full pressure sampling tube of one set of flue gas multipoint flow rate sampling probes, and the second full pressure sampling tube solenoid valve set is connected with the full pressure sampling tube of another set of the flue gas multipoint flow rate sampling probes; wherein the first static pressure sampling tube solenoid valve set is connected with the static pressure sampling tube of one set of flue gas multipoint flow rate sampling probes, and the second static pressure sampling tube solenoid valve set is connected with the static pressure sampling tube of another set of flue gas multipoint flow rate sampling probes.

10. The online monitoring for flue gas carbon dioxide emissions system according to claim 1, wherein the CO2 concentration analyzer is an in-situ hot moisture CO2 concentration analyzer, and the temperature-pressure-humidity analyzer is an integrated temperature-pressure-humidity analyzer.
